(19) United States
(12) Patent Application Publication
(10) Pub. No.: US 2015/0238145 A1
(43) Pub. Date: Aug. 27, 2015

(54) MOUSE DEVICE

(71) Applicant: PRIMAX ELECTRONICS LTD., NEIHU (TW)

(72) Inventor: SHIH-CHIEH LAN, TAIPEI (TW)

(73) Assignee: PRIMAX ELECTRONICS LTD., NEIHU (TW)

(21) Appl. No.: 14/286,681

(22) Filed: May 23, 2014

(30) Foreign Application Priority Data
Feb. 27, 2014 (CN) 201410067780.1

(51) Int. Cl. A61B 5/00; A61B 5/024; A61B 5/026; G06F 3/0354; H04N 5/33

(52) U.S. Cl. CPC A61B 5/6897; G06F 3/03543; H04N 5/33; A61B 5/0261; A61B 5/02433; A61B 5/7278; A61B 5/7282; A61B 5/746; A61B 5/7405; A61B 5/7455; A61B 5/7445; A61B 5/486; A61B 5/004; A61B 2560/0462; A61B 2562/0238; A61B 2576/02

(57) ABSTRACT

A mouse device includes a grip part, an extension part, an infrared light sensing unit, and a controlling unit. The extension part is extended from the grip part for supporting a wrist of a user. The infrared light sensing unit includes an infrared light source and an image sensor. The infrared light source emits an infrared light beam. A portion of the infrared light beam within a specified wavelength range is absorbed by the plural blood vessels of the wrist. Another portion of the infrared light beam beyond the specified wavelength range is reflected from the plural blood vessels to the image sensor, so that plural infrared images are generated. The controlling unit calculates a blood flow rate of the plural blood vessels of the wrist according to the plural infrared images, and acquires the body information of the user according to the blood flow rate.

### FIELD OF THE INVENTION

The present invention relates to a mouse device, and more particularly to a mouse device capable of detecting a body information.

### BACKGROUND OF THE INVENTION

Generally, a mouse device is widely operated to control an electronic device such as a computer. With the popularity of the electronic device and the lifestyle change, the electronic device is frequently operated by the user. After the mouse device has been operated for a long time period, the user may be suffered from fatigue. However, it is difficult for the user to be aware of the fatigue condition during the process of operating the mouse device. If no proper mechanism is provided to detect the body information of the user and warn the user of taking a rest or taking medical treatment according to the body information, the accumulated long-term fatigue may discomfort the user or even disease the user. Therefore, there is a need of providing an improved mouse device in order to overcome the above drawbacks.

### SUMMARY OF THE INVENTION

The present invention relates to a mouse device having a function of avoiding excessive fatigue of the user.

In accordance with an aspect of the present invention, there is provided a mouse device. The mouse device includes a grip part, an extension part, an infrared light sensing unit, and a controlling unit. The grip part is gripped by a hand of a user. The extension part is extended from the grip part, and supports a wrist of the user. The infrared light sensing unit is disposed on the extension part, and detects a body information of the user. The infrared light sensing unit includes an infrared light source and an image sensor. The infrared light source emits an infrared light beam that is absorbable by plural blood vessels of the wrist. When the infrared light beam from the infrared light source is projected on the wrist, a first portion of the infrared light beam within a specified wavelength range is absorbed by the plural blood vessels of the wrist, and a second portion of the infrared light beam beyond the specified wavelength range is reflected from the plural blood vessels. After the second portion of the infrared light beam reflected from the plural blood vessels is received by the image sensor, plural infrared images are generated. The controlling unit is electrically connected with the infrared light sensing unit, and receiving the plural infrared images. The controlling unit calculates a blood flow rate of the plural blood vessels of the wrist according to the plural infrared images, and acquires the body information of the user according to the blood flow rate.

The above objects and advantages of the present invention will become more readily apparent to those ordinarily skilled in the art after reviewing the following detailed description and accompanying drawings, in which:

### BRIEF DESCRIPTION OF THE DRAWINGS

FIG. 1 is a schematic view illustrating the outer appearance of a mouse device according to an embodiment of the present invention;

FIG. 2 is schematic functional block diagram illustrating the mouse device according to the embodiment of the present invention;

FIG. 3 schematically illustrates the mouse device in a usage status according to the embodiment of the present invention; and

FIG. 4 schematically illustrates the infrared light sensing unit used in the mouse device according to the embodiment of the present invention.

### DETAILED DESCRIPTION OF THE PREFERRED EMBODIMENT

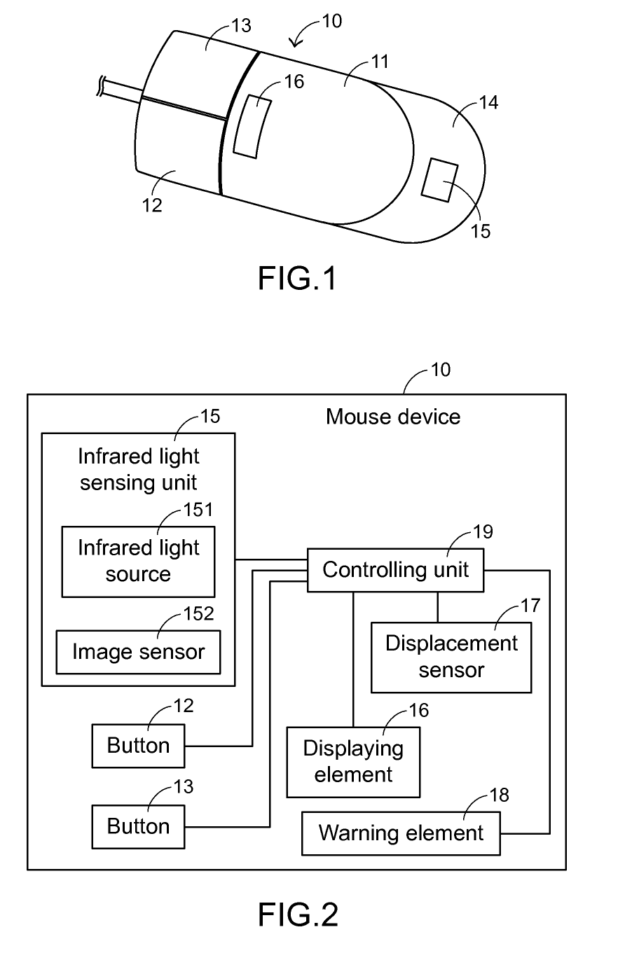

Please refer to FIGS. 1 and 2. FIG. 1 is a schematic view illustrating the outer appearance of a mouse device according to an embodiment of the present invention. FIG. 2 is schematic functional block diagram illustrating the mouse device according to the embodiment of the present invention. As shown in FIGS. 1 and 2, the mouse device 10 comprises a grip part 11, two buttons 12, 13, an extension part 14, an infrared light sensing unit 15, a displaying element 16, a displacement sensor 17, a warning element 18, and a controlling unit 19. The infrared light sensing unit 15 comprises an infrared light source 151 and an image sensor 152. The buttons 12, 13, the infrared light sensing unit 15, the displaying element 16, the displacement sensor 17 and the warning element 18 are electrically connected with the controlling unit 19.

In this embodiment, the mouse device 10 is a wired mouse device or a wireless mouse device. In case that the mouse device 10 is the wired mouse device, the mouse device 10 may be in communication with the corresponding electronic device through a USB connecting wire, a Micro USB connecting wire or any other well-known connecting wire. In case that the mouse device 10 is the wireless mouse device, the mouse device 10 may be in communication with the corresponding electronic device by a radio frequency communication technology, an infrared communication technology, a Bluetooth communication technology, an IEEE 802.11 communication technology or any other appropriate wireless communication technology. Hereinafter, the mouse device 10 will be illustrated by referring to the wired mouse device.

As shown in FIG. 2, the displacement sensor 17 is disposed within the mouse device 10 for detecting a moving direction and a displacement amount of the mouse device 10. Consequently, the user may move the mouse device 10 to change a coordinate position of a cursor displayed on a computer monitor (not shown), which is connected with the mouse device 10. The displacement sensor 17 may be a well-known mouse displacement sensor such as an infrared sensor or a laser sensor. The functions and the operating principles of the displacement sensor 17 are similar to those of the well-known mouse displacement sensor, and are not redundantly described herein.

Please refer to FIGS. 1 and 2 again. The grip part 11 is disposed on a top surface of the mouse device 10. A user's hand (especially a user's palm) may be placed on the grip part 11. When the grip part 11 of the mouse device 10 is gripped by the users' hand, the mouse device 10 can be smoothly operated by the user. The two buttons 12 and 13 are located at a front end of the grip part 11. Moreover, the two buttons 12 and 13 are arranged side by side. When one of the two buttons 12 and 13 is depressed, the depressed button 12 or 13 generates a command to the controlling unit 19 in order to control the computer. For example, a left button command of the well-known mouse device is generated when the button 12 is depressed, and a right button command of the well-known mouse device is generated when the button 13 is depressed. In this embodiment, the grip part 11 is a top cover of the mouse device 10. It is noted that numerous modifications and alterations may be made while retaining the teachings of the invention. For example, in some other embodiments, the grip part 11 may be separated from the mouse device through a well-known detachable connecting structure (e.g. a Velcro fastener or an engaging mechanism). Consequently, the grip part 11 may be kept clean, and the tactile feel of gripping the grip part 11 will be enhanced. Moreover, the grip part 11 is made of a high friction material such as rubber.

Moreover, the top surface of the mouse device 10 may be equipped with additional buttons, a touch panel or a scroll wheel in order to generate additional commands, a touch signal or a scrolling signal for scrolling a window. Consequently, the sensitivity (e.g. dots per linear inch) of the mouse device 10 may be adjusted, the movement of the cursor may be controlled, or the scroll bars of the window of the computer may be scrolled along an upward/downward direction or a leftward/rightward direction.

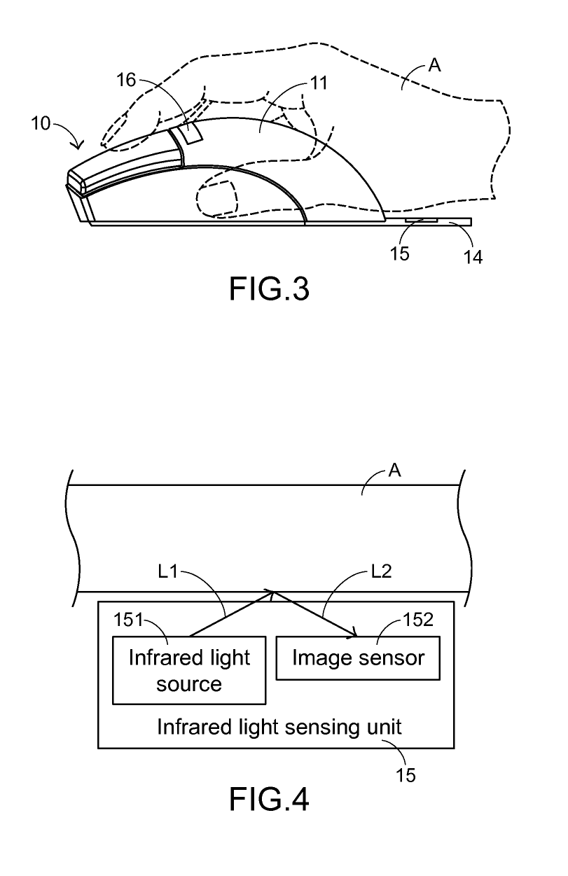

Please also refer to FIG. 3. FIG. 3 schematically illustrates the mouse device in a usage status according to the embodiment of the present invention. As shown in FIG. 3, the extension part 14 is extended from a rear end of the grip part 11 for supporting a user's wrist A. In an embodiment, the extension part 14 is directly fixed on the rear end of the grip part 11. Alternatively, in some other embodiments, an accommodation space (not shown) is formed in the rear end or the bottom of the mouse device 10 for accommodating the extension part 14. In particular, the extension part 14 may be stored within the accommodation space or protruded from the accommodation space through a sliding track, a rotating mechanism, a folding mechanism or any other appropriate storing mechanism. Consequently, the extension part 14 may be protruded from the accommodation space or the protruded distance or the angle of the extension part 14 may be adjusted according to the practical requirements. In this embodiment, the extension part 14 is directly fixed on the rear end of the grip part 11.

An example of the extension part 14 includes but is not limited to a rigid plate or a soft plate. Moreover, the exterior shape of the extension part 14 may be specially designed. For example, the extension part 14 may be an internally-concaved curvy plate that matches the profile of the user's wrist A, or the extension part 14 may be an externally-convex curvy plate to be sustained against the user's wrist A.

The infrared light sensing unit 15 is installed on the extension part 14 for detecting a body information of the user. In this embodiment, the infrared light sensing unit 15 is embedded into a top surface of the extension part 14, but is not limited thereto. When the mouse device 10 is operated by the user in the normal hand gesture, the user's wrist A is located over the infrared light sensing unit 15 or directly contacted with a top surface of the infrared light sensing unit 15. The operating principle of using the infrared light sensing unit 15 to detect the body information of the user will be illustrated as follows.

FIG. 4 schematically illustrates the infrared light sensing unit used in the mouse device according to the embodiment of the present invention. As shown in FIG. 4, the infrared light sensing unit 15 comprises the infrared light source 151 and the image sensor 152. The infrared light source 151 and the image sensor 152 are disposed within the infrared light sensing unit 15. In an embodiment, the infrared light source 151 is a well-known infrared light emitting diode. The infrared light source 151 may emit an infrared light beam L1 to the user's wrist A. The infrared light beam L1 has a wavelength in the range between 700 nanometers and 10 millimeters. An example of the image sensor 152 is a well-known charge coupled device (CCD). The image sensor 152 may receive a reflected infrared light beam L2 from the user's wrist A and generate an infrared image according to the reflected infrared light beam L2.

In particular, the blood of the blood vessel of the human body contains hemoglobin, and the portion of the infrared light beam within a specified wavelength range (e.g. between 700 nanometers and 1000 nanometers) may be absorbed by hemoglobin. Consequently, when the infrared light beam L1 with the wavelength in the range between 700 nanometers and 10 millimeters is projected on the user's wrist A, the portion of the infrared light beam within the wavelength range between 700 nanometers and 1000 nanometers is absorbed by plural blood vessels of the user's wrist A. On the other hand, the infrared light beam L2 beyond the wavelength range between 700 nanometers and 1000 nanometers cannot be absorbed by the plural blood vessels of the user's wrist A. Consequently, the infrared light beam L2 is reflected to the image sensor 152, and then received by the image sensor 152.

When the heart of the user contracts, the heart pumps out the blood to the artery blood vessel, and pushes the blood in the artery blood vessel to flow. Consequently, the flow rate of the blood in the artery increases. On the other hand, when the heart relaxes, the blood is no longer pushed by the heart. Consequently, the flow rate of the blood in the artery decreases. After the infrared light beam L2 reflected from the user's wrist A is received by the image sensor 152, the image sensor 152 generates n infrared images per seconds. Moreover, in case that the blood flow rates in the artery are different, the images of the artery blood vessels contained in these infrared images are different.

Next, the plural infrared images are sequentially transmitted from the image sensor 152 to the controlling unit 19. Please refer to FIG. 2. After the plural infrared images from the image sensor 152 are received by the controlling unit 19, the plural infrared images are analyzed by the controlling unit 19 according to the well-known image recognition method. Consequently, a blood flow rate of the plural blood vessels in the user's wrist A is calculated by the controlling unit 19, and the body information of the user is acquired by the controlling unit 19 according to the blood flow rate. In an embodiment, the body information is a pulsation information. The larger n value indicates that the image sensor 152 generates more infrared images per seconds. Under this circumstance, the sensitivity of the infrared light sensing unit 15 is increased, and the precision of calculating the pulsation information by the controlling unit 19 is enhanced.

After the controlling unit 19 calculates the blood flow rate of the user according to the plural infrared images from the infrared light sensing unit 15 and acquires the body information of the user, the controlling unit 19 issues a displaying information to the displaying element 16. Please refer to FIGS. 1 and 2 again. The displaying element 16 is disposed on the grip part 11 and electrically connected with the controlling unit 19. The body information may be displayed on the displaying element 16. Since the body information in this embodiment is the pulsation information, the number of heartbeat of the user per minute may be displayed on the displaying element 16. Moreover, an example of the displaying element 16 includes but is not limited to a well-known liquid crystal display.

The warning element 18 is disposed within the mouse device 10 or located at a side of the mouse device 10. The warning element 18 may generate a sound effect, a vibration effect or a light change. After the body information of the user is acquired by the controlling unit 19, the controlling unit 19 will automatically judge whether the body information complies with a standard body information. For example, the number of heartbeat of a normal adult is in a standard range between 60 and 100 beats per minutes. The controlling unit 19 may judge whether the pulsation information is higher or lower than the standard range.

If the pulsation information is higher or lower than the standard range, the controlling unit 19 issues a warning signal to the warning element 18. In response to the warning signal, the warning element 18 generates a sound effect, a vibration effect or a light change to warn the user of paying attention to her (or his) body health. The warning element 18 is for example a speaker, a vibration motor or a LED lamp. Moreover, according to the warning characteristics of the warning element 18, the warning element 18 may be located at a proper position of the mouse device 10. For example, in case that the warning element 18 is the LED lamp, the warning element 18 is installed on the top cover of the mouse device 10 in order to facilitate the user to visually observe the light change. In this embodiment, the warning element 18 is the vibration motor, and the warning element 18 is installed within the mouse device 10. If the pulsation information is higher or lower than 60 and 100 beats per minutes, the warning element 18 may vibrate the grip part 11 to warn the user.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A mouse device, comprising:
a grip part to be gripped by a hand of a user;
an extension part extended from the grip part, and supporting a wrist of the user;
an infrared light sensing unit disposed on the extension part, and detecting a body information of the user, wherein the infrared light sensing unit comprises:
an infrared light source emitting an infrared light beam that is absorbable by plural blood vessels of the wrist, wherein when the infrared light beam from the infrared light source is projected on the wrist, a first portion of the infrared light beam within a specified wavelength range is absorbed by the plural blood vessels of the wrist, and a second portion of the infrared light beam beyond the specified wavelength range is reflected from the plural blood vessels; and
an image sensor, wherein after the second portion of the infrared light beam reflected from the plural blood vessels is received by the image sensor, plural infrared images are generated; and
a controlling unit electrically connected with the infrared light sensing unit, and receiving the plural infrared images, wherein the controlling unit calculates a blood flow rate of the plural blood vessels of the wrist according to the plural infrared images, and acquires the body information of the user according to the blood flow rate.

2. The mouse device according to claim 1, wherein the body information is a pulsation information.

3. The mouse device according to claim 1, further comprising a displaying element, wherein the displaying element is disposed on the grip part, and the body information of the user is displayed on the displaying element.

4. The mouse device according to claim 1, further comprising a warning element, wherein if the body information is higher or lower than a standard range, the warning element generates a sound effect, a vibration effect or a light change.

5. The mouse device according to claim 1, further comprising:
a displacement sensor detecting a displacement of the grip part; and
two buttons, wherein when one of the two buttons is depressed, a corresponding command is generated,
wherein the displacement sensor and the two buttons are electrically connected with the controlling unit.
